(19) United States
(12) Patent Application Publication
(10) Pub. No.: US 2018/0333415 A1
(43) Pub. Date: Nov. 22, 2018

(54) THERAPEUTIC METHODS

(71) Applicant: REGENTS OF THE UNIVERSITY OF MINNESOTA, Minneapolis, MN (US)

(72) Inventors: David A. Potter, Minneapolis, MN (US); Zhijun Guo, Minneapolis, MN (US)

(21) Appl. No.: 15/984,092

(22) Filed: May 18, 2018

Related U.S. Application Data
(60) Provisional application No. 62/508,900, filed on May 19, 2017.

(51) Int. Cl. A61K 31/517; A61K 45/06; A61P 35/00; A61P 31/00

(52) U.S. Cl. CPC A61K 31/517; A61P 31/00; A61P 35/00; A61K 45/06

(57) ABSTRACT

Disclosed are therapeutic methods for treating cancer such as breast cancer.

### PRIORITY

This application claims priority to U.S. Provisional Patent Application Number 62/508,900, filed May 19, 2017. The entire content of the application referenced above is hereby incorporated by reference herein.

### GOVERNMENT FUNDING

This invention was made with government support under 2P30CA077598 awarded by the National Cancer Institute and 5UL1TR000114 awarded by the National Center for Advancing Translational Sciences. The government has certain rights in the invention.

### BACKGROUND

Poziotinib is a quinazoline-based receptor tyrosine kinase inhibitor that has been investigated in the inhibition of HER2 positive (HER2+) breast cancer, gastric cancer and other HER2+ malignancies. Poziotinib 1-(4-((4-((3,4-dichloro-2-fluorophenyl)amino)-7-methoxyquinazolin-6-yl)oxy)piperidin-1-yl)prop-2-en-1-one) has been described in WO2008/150118 and has the structure of formula I:

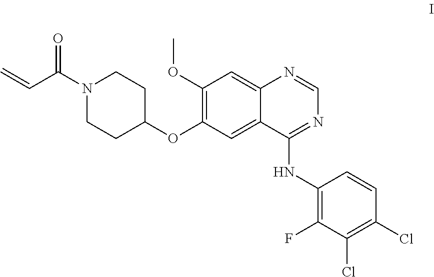 [SMILES: C=CC(=O)N1CCC(Oc2cc3c(Nc4ccc(Cl)c(Cl)c4F)ncnc3cc2OC)CC1]

Poziotinib was shown to inhibit EGFR type I kinase and inhibit cell growth in various cancer cell lines including colon/rectal, skin, lung and the breast cancer cell line SK-BR3 that overexpresses the HER2 gene product (WO2008/150118).

Approximately 60% of human breast cancers are ER positive HER2 negative (ER+HER2−) related breast cancers. Accordingly, there is a significant need for chemotherapeutic (e.g. cytotoxic) agents to treat cancers including breast cancers such as ER+HER2− breast cancer.

### SUMMARY OF THE INVENTION

Applicant has discovered that poziotinib 1-(4-((4-((3,4-dichloro-2-fluorophenyl)amino)-7-methoxyquinazolin-6-yl)oxy)piperidin-1-yl)prop-2-en-1-one or a pharmaceutically acceptable salt thereof), a HER2 tyrosine kinase inhibitor, potently inhibits the growth of ER positive HER2 negative (ER+HER2−) breast cancer cells. The discovery of this activity was significant because ER+HER2− breast cancer may lack the HER2 target as well as erbB1 and erbB4 kinases. Poziotinib may exert part of this anticancer effect by inhibiting cytochrome P450 arachidonic acid (AA) epoxygenase enzymes, which synthesize epoxyeicosatrienoic acids (EETs), thus inhibiting ER+HER2− breast cancer which depends on CYP3A4 and CYP2C8 as well as related CYP3A and CYP2C family enzymes for growth. Furthermore, triple negative breast cancer may depend on CYP3A and CYP2C AA enzymes for growth. Accordingly, poziotinib may be useful as an inhibitor of ER+HER2− breast cancer through inhibition of CYP3A4 and CYP2C8 enzyme family epoxygenase activity and triple negative breast cancer through inhibition of CYP3A4 and CYP2C8 and related CYP3A and CYP2C enzyme families. The use of poziotinib may also allow the inhibition of ER+HER2− and triple negative breast cancer through a novel mechanism of action and provide non-cross resistant therapy for this type of breast cancer. Enhancement of chemotherapy by the combination of CYP3A4 inhibition with poziotinib and other agents such as paclitaxel may be possible.

Unlike neratinib that inhibits ER negative HER2 positive breast cancer (Park, I. W., The New England Journal of Medicine. 2016, 375(1), 11-22), poziotinib inhibits ER positive breast cancer and thus may also inhibit ER positive HER2 positive breast cancer. Further combination of poziotinib with an immune checkpoint inhibitor may reduce oxygen consumption of the epithelial component of tumors resulting in enhanced cytotoxic T cell activity which may also provide useful anticancer therapy (Cancer Immunol Res. 2017 Jan; 5(1): 9-16).

Accordingly, one embodiment provides a method for treating ER positive HER2 negative (ER+HER2−) breast cancer or triple negative breast cancer in a human patient with ER+HER2− breast cancer or triple negative breast cancer comprising administering to the human patient in need thereof an effective amount of poziotinib.

One embodiment provides a method for treating ER positive HER2 negative (ER+HER2−) breast cancer or triple negative breast cancer in a human patient with ER+HER2− breast cancer or triple negative breast cancer comprising (a) determining the HER2 status of the breast cancer in the human patient and (b) administering to the human patient in need thereof an effective amount of poziotinib.

One embodiment provides a method for treating ER positive HER2 negative (ER+HER2−) breast cancer, triple negative breast cancer, or ER positive HER2 positive (ER+HER2+) breast cancer in a human patient with ER+HER2− breast cancer, triple negative breast cancer, or ER+HER2+ breast cancer comprising administering to the human patient in need thereof an effective amount of poziotinib.

One embodiment provides a method for treating ER positive HER2 negative (ER+HER2−) breast cancer, triple negative breast cancer, or ER positive HER2 positive (ER+HER2+) breast cancer in a human patient with ER+HER2− breast cancer, triple negative breast cancer, or ER+HER2+ breast cancer comprising (a) determining the HER2 status of the breast cancer in the human patient and (b) administering to the human patient in need thereof an effective amount of poziotinib.

### BRIEF DESCRIPTION OF THE FIGURES

FIG. 1 shows the effect of poziotinib on MCF-7 proliferation in complete media.

FIG. 2 shows the effect of poziotinib on CYP2C8 supersome-mediated EET biosynthesis.

FIG. 3 shows the effect of poziotinib on CYP3A4 supersome-mediated EET biosynthesis.

FIG. 4 shows OCR and ECAR after MCF7 treatment with poziotinib (40 and 80 uM) vs. DMSO.

### DETAILED DESCRIPTION

The term “treatment” or “treating,” to the extent it relates to a disease or condition includes inhibiting the disease or condition, eliminating the disease or condition, and/or relieving one or more symptoms of the disease or condition.

The term “patient” as used herein refers to any animal including mammals such as humans, higher non-human primates, rodents domestic and farm animals such as cow, horses, dogs and cats. In one embodiment, the patient is a human patient (e.g., a human female patient).

The phrase “therapeutically effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

While certain methods described herein refer to poziotinib, it is to be understood that such methods include pharmaceutically acceptable salts of 1-(4-((4-((3,4-dichloro-2-fluorophenyl)amino)-7-methoxyquinazolin-6-yl)oxy)piperidin-1-yl)prop-2-en-1-one).

Poziotinib may also be used in combination with one or more additional hormonal agents, immunotherapeutic agents, chemotherapeutic (e.g. cytotoxic) agents or agents that inhibit the biosynthesis of one or more epoxyeicosatrienoic acids (EETs) for preventing or treating occurrence or recurrence of ER+HER2− breast cancer, triple negative breast cancer, or ER+HER2+ breast cancer.

One embodiment provides a pharmaceutical composition comprising poziotinib and one or more additional hormonal agents, immunotherapeutic agents, chemotherapeutic (e.g. cytotoxic) agents, or agents that inhibit the biosynthesis of one or more epoxyeicosatrienoic acids (EETs) for preventing or treating occurrence or recurrence of ER+HER2− breast cancer, triple negative breast cancer, or ER+HER2+ breast cancer.

In at least one embodiment, the cytotoxic agent is selected from the group consisting of an antimetabolite, a mitotic inhibitor, alkylating agent, a platinum-based antineoplastic drug, an mTOR inhibitor, a VEGF inhibitor, an aromatase inhibitor and a CDK4/6 inhibitor.

In another embodiment, poziotinib may be administered with an antimetabolite that is selected from the group consisting of Capecitabine, 5-Fluorouracil, Gemcitabine, Pemetrexed, Methotrexate, 6-Mercaptopurine, Cladribine, Cytarabine, Doxifludine, Floxuridine, Fludarabine, Hydroxycarbamide, decarbazine, hydroxyurea, and asparaginase.

In other embodiments, mitotic inhibitor may be a microtubule-destabilizing agent, a microtubule-stabilizing agent, or a combination thereof. The mitotic inhibitor may be taxane, vinca alkaloid, epothilone, or a combination thereof. The mitotic inhibitor may also be selected from BT-062, HMN-214, eribulin mesylate, vindesine, EC-1069, EC-1456, EC-531, vintafolide, 2-methoxyestradiol, GTx-230, trastuzumab emtansine, crolibulin, D1302A-maytansinoid conjugates IMGN-529, lorvotuzumab mertansine, SAR-3419, SAR-566658, IMP-03138, topotecan/vincristine combinations, BPH-8, fosbretabulin tromethamine, estramustine phosphate sodium, vincristine, vinflunine, vinorelbine, RX-21101, cabazitaxel, STA-9584, vinblastine, epothilone A, patupilone, ixabepilone, Epothilone D, paclitaxel, docetaxel, DJ-927, discodermolide, eleutherobin, and pharmaceutically acceptable salts thereof or combinations thereof.

As used herein, an “alkylating agent” is a substance that adds one or more alkyl groups (CnHm, where n and m are integers) to a nucleic acid. In the present invention, an alkylating agent is selected from the group consisting of nitrogen mustards, nitrosoureas, alkyl sulfonates, triazines, ethylenimines, and combinations thereof. Non-limiting examples of nitrogen mustards include mechlorethamine, chlorambucil, cyclophosphamide, bendamustine, ifosfamide, melphalan, melphalan flufenamide, and pharmaceutically acceptable salts thereof. Non-limiting examples of nitrosoureas include streptozocin, carmustine, lomustine, and pharmaceutically acceptable salts thereof. Non-limiting examples of alkyl sulfonates include busulfan and pharmaceutically acceptable salts thereof. Non-limiting examples of triazines include dacarbazine, temozolomide, and pharmaceutically acceptable salts thereof. Non-limiting examples of ethylenimines include thiotepa, altretamine, and pharmaceutically acceptable salts thereof. Other alkylating agents include ProLindac, Ac-225 BC-8, ALF-2111, trofosfamide, MDX-1203, thioureidobutyronitrile, mitobronitol, mitolactol, nimustine, glufosfamide, HuMax-TAC and PBD ADC combinations, BP-C1, treosulfan, nifurtimox, improsulfan tosilate, ranimustine, ND-01, HH-1, 22P1G cells and ifosfamide combinations, estramustine phosphate, prednimustine, lurbinectedin, trabectedin, altreatamine, SGN-CD33A, fotemustine, nedaplatin, heptaplatin, apaziquone, SG-2000, TLK-58747, laromustine, procarbazine, and pharmaceutically acceptable salts thereof.

The platinum-based antineoplastic drug may be for example, Cisplatin, Carboplatin, Dicycloplatin, Eptaplatin, Lobaplatin, Miriplatin, Nedaplatin, Oxaliplatin, Picoplatin, or Satraplatin.

The term “mTOR inhibitors (mTOR inhibitor)” as used herein is used for purposes of a material to inhibit the mTOR signaling pathway of the conventional anticancer agents or immunosuppressive agents. The mTOR inhibitor may be rapamycin, temsirolimus, everolimus, ridaforolimus, MLN4924, XL388, GDC-0349, AZD2014, AZD8055, GSK105965, MLN0128 Ridaforlimus and the like.

“VEGF inhibitor” as used herein is any substance that decreases signaling by the VEGF-VEGFR pathway. VEGF inhibitors can be, to name just a few examples, small molecules, peptides, polypeptides, proteins, including more specifically antibodies, including anti-VEGF antibodies, anti-VEGFR antibodies, intrabodies, maxibodies, minibodies, diabodies, Fc fusion proteins such as peptibodies, receptibodies, soluble VEGF receptor proteins and fragments, and a variety of others. Many VEGF inhibitors work by binding to VEGF or to a VEGF receptor. Others work more indirectly by binding to factors that bind to VEGF or to a VEGF receptor or to other components of the VEGF signaling pathway. Still other VEGF inhibitors act by altering regulatory posttranslational modifications that modulate VEGF pathway signaling. VEGF inhibitors in accordance with the invention also may act through more indirect mechanisms. Whatever the mechanism involved, as used herein, a VEGF inhibitor decreases the effective activity of the VEGF signaling pathway in a given circumstance over what it would be in the same circumstance in the absence of the inhibitor.

Non-limiting examples of VEGF inhibitors include: (a) 4TBPPAPC or a closely related compound described in U.S. 2003/0125339 or U.S. Pat. No. 6,995,162 which is herein incorporated by reference in its entirety, particularly in parts disclosing 4TBPPAPC and closely related VEGF inhibitors; (b) AMG 706 or a closely related substituted alkylamine derivative described in U.S. 2003/0125339 or U.S. 2003/0225106 or U.S. Pat. No. 6,995,162 or U.S. Pat. No. 6,878,714 each of which is herein incorporated by reference in its entirety, particularly in parts disclosing AMG 706 and these closely related VEGF inhibitors; (c) Avastin™ or a closely related non-naturally occurring humanized monoclonal antibody that binds to VEGF, is a VEGF inhibitor, and is at least 90% identical in sequence to Avastin™; (d) Nexavar® or a closely related substituted omega-carboxyaryl diphenyl urea or derivative thereof described in WO00/42012, WO00/41698, U.S. 2005/0038080A1, U.S. 2003/0125359A1, U.S. 2002/0165394A1, U.S. 2001/003447A1, U.S. 2001/0016659A1, and U.S. 2002/013774A1 which are herein incorporated by reference in their entirety, particularly in parts disclosing these VEGF inhibitors; (e) PTK/ZK or a closely related anilinophthalazine or derivative thereof that binds to and inhibits the activity of multiple receptor tyrosine kinases including binding to the protein kinase domain and inhibition of VEGFR1 and VEGFR2; (f) Sutent® or a closely related derivative of (5-[5-fluoro-2-oxo-1,2-dihydroindol-(3Z)-ylidenemethyl]-2,4-dimethyl-1H- pyrrole-3-carboxylic acid [2-diethylaminoethyl]amide) that is a VEGF inhibitor; and (g) VEGFinhibitors as described in U.S. 2006/0241115, including those of Formula IV therein.

Further examples of VEGF inhibitors are the following: (a) 4TBPPAPC, as described in U.S. 2003/0125339 or U.S. Pat. No. 6,995,162 which is herein incorporated by reference in its entirety, particularly in parts disclosing 4TBPPAPC; (b) AMG 706, as described in U.S. 2003/0125339 or U.S. Pat. No. 6,995,162 or U.S. Pat. No. 6,878,714 which is herein incorporated by reference in its entirety, particularly in parts disclosing AMG 706; (c) Avastin™; (d) Nexavar®, as described in WO00/42012, WO00/41698, U.S. 2005/0038080A1, U.S. 2003/0125359A1, U.S. 2002/0165394A1, U.S. 2001/003447A1, U.S. 2001/0016659A1, and U.S. 2002/013774A1 which are herein incorporated by reference in their entirety, particularly in parts disclosing Nexavar®; (e) PTK/ZK; (f) Sutent®, and (g) VEGF inhibitors of Formula IV as described in U.S. 2006/0241115.

In some embodiments, the VEGF inhibitor is pegaptanib. In one embodiment, the VEGF inhibitor is bevacizumab. In one embodiment, the VEGF inhibitor is ranibizumab. In one embodiment, the VEGF inhibitor is lapatinib. In one embodiment, the VEGF inhibitor is sorafenib. In one embodiment, the VEGFinhibitor is sunitinib. In one embodiment, the VEGF inhibitor is axitinib. In one embodiment, the VEGF inhibitor is pazopanib. In one embodiment, the VEGFinhibitor is aflibercept.

By “aromatase inhibitor” it is meant non-steroidal and steroidal compounds that inhibit the enzyme aromatase thereby preventing the conversion of androgens to estrogens, preferably those which inhibit aromatase activity in vitro with an IC50 value of less than 10-5 M as well as their pharmaceutically acceptable salts. Exemplary aromatase inhibitors for use in the methods herein described include without limitation anastrozole, letrozole, exemestane, vorozole, formestane, fadrozole, aminoglutethimide, testolactone, 4-hydroxyandrostenedione, 1,4,6-androstatrien-3,17-dione and 4-androstene-3,6,17-trione.

The terms “cyclin dependent kinase 4/6 inhibitor” and “CDK4/6 inhibitor” as used herein refer to a compound that selectively targets, decreases, or inhibits at least one activity of CDK4 and/or CDK6. Non-limiting examples of inhibitors of CDK4/6 include Abemaciclib (LY2835219), palbociclib (PD0332991), LEE-011 (ribociclib), LY2835219 (abemaciclib), G1T28-1, SHR6390, or P276-00, or a derivative of any one of palbociclib, LEE-011, G1T28-1, SHR6390, or P276-00. In certain embodiments, the CDK4/6 inhibitor may be derived from pyridopyrimidine, pyrrolopyrimidine or indolocarbazole compounds.

As used herein, a “molecularly targeted agent” is a substance that interferes with the function of a single molecule or group of molecules, preferably those that are involved in tumor growth and progression, when administered to a subject. Non-limiting examples of molecularly targeted agent of the present invention include signal transduction inhibitors, modulators of gene expression and other cellular functions, immune system modulators, antibody-drug conjugates (ADCs), and combinations thereof.

The molecularly targeted agent may be selected from epidermal growth factor receptor family inhibitors (EGFRi), mammalian target of rapamycin (mTor) inhibitors, immune checkpoint inhibitors, anaplastic lymphoma kinase (ALK) inhibitors, B-cell lymphoma-2 (BCL-2) inhibitors, B-Raf inhibitors, cyclin-dependent kinase inhibitors (CDKi), ERK inhibitors, histone deacetylase inhibitors (HDACi), heat shock protein-90 inhibitors (HSP90i), Janus kinase inhibitors, mitogen activated protein kinase (MAPK) inhibitors, MEK inhibitors, poly ADP ribose polymerase (PARP) inhibitors, phosphoinositide 3-kinase inhibitors (PI3Ki), Ras inhibitors, and combinations thereof.

The molecularly targeted agent may be selected from ado-trastuzumab emtansine, alemtuzumab, cetuximab, ipilimumab, ofatumumab, panitumumab, pertuzumab, rituximab, tositumomab, 131I-tositumomab, trastuzumab, brentuximab vedotin, denileukin diftitox, ibritumomab tiuxetan, axitinib, bortezomib, bosutinib, cabozantinib, crizotinib, carfilzomib, dasatinib, erlotinib, gefitinib, imatinib mesylate, lapatinib, nilotinib, pazopanib, ponatinib, regorafenib, ruxolitinib, sorafenib, sunitinib, tofacitinib, vandetanib, vemurafenib, alitretinoin, bexarotene, everolimus, romidepsin, temsirolimus, tretinoin, vorinostat, and pharmaceutically acceptable salts thereof or combinations thereof. The molecularly targeted agent may include an antibody or an antibody moiety.

In other combinations, the patient may receive a second EGFR inhibitor such as erlotinib, gefitinib, lapatinib, canetinib, pelitinib, neratinib, (R,E)-N-(7-chloro-1-(1-(4-(dimethylamino)but-2-enoyl)azepan-3-yl)-1H-benzo[d]imidazol-2-yl)-2-methylisonicotinamide, Trastuzumab, Margetuximab, panitumumab, matuzumab, Necitumumab, pertuzumab, nimotuzumab, zalutumumab, Necitumumab, cetuximab, icotinib, afatinib, and pharmaceutically acceptable salt thereof. The molecularly targeted agent may be an anti-EGFR family antibody or a complex including the anti-EGFR family antibody. The anti-EGFR family antibody may be an anti-HER1 antibody, an anti-HER2 antibody, or an anti-HER4 antibody.

In one embodiment the chemotherapeutic agent is a breast cancer chemotherapeutic agent.

In one embodiment the cytotoxic agent is a breast cancer cytotoxic agent.

In one embodiment the chemotherapeutic (e.g. cytotoxic)agents are independently selected from a selective estrogen receptor modifier, an aromatase inhibitor, a taxane, a epothilone, a halochondrin, a platin, a vinca alkaloid, a cyclophosphamide, an alkylating agent, a CDK4 inhibitor, a CDK6 inhibitor, a mTOR inhibitor, and a HER2 targeted agent.

In one embodiment the chemotherapeutic (e.g. cytotoxic) agents are independently selected from tamoxifen, fulvestrant, raloxifene, anastrozole, letrozole, exemestane, paclitaxel, docetaxel, ixabepilone, eribulin, capecitabine, gemcitabine, vinorelbine, palbociclib, ribociclib, everolimus, trastuzumab, pertuzumab, and lapatinib.

In one embodiment the hormonal agent is tamoxifen or an aromatase inhibitor.

In one embodiment the hormonal agent is tamoxifen and the patient is pre-menopausal or post-menopausal.

In one embodiment the compound that inhibits the biosynthesis of one or more epoxyeicosatrienoic acids (EETs) for use as an agent in addition to poziotinib is a compound described in US Patent Publication U.S. 2015/0342909 which document is hereby incorporated by reference in its entirety (e.g., all compounds of formula I or a pharmaceutically acceptable salt thereof). In one embodiment the compound that inhibits the biosynthesis of one or more epoxyeicosatrienoic acids (EETs) is N1-hexyl-N5-benzyl biguanide or a pharmaceutically acceptable salt thereof (e.g., N1-hexyl-N5-benzyl biguanide hydrochloride) or metformin.

In one embodiment the immune checkpoint targeted therapy is a PD-1 antibody such as pembrolizumab or nivolumab, a PD-L1 antibody such as atezolizumab or avelumab or a small molecule blocker for PD-1 ligand interactions.

In one embodiment the immune checkpoint targeted therapy is a CTLA4 antibody such as ipilimumab.

In one embodiment the hormonal agent is an aromatase inhibitor and the patient is post-menopausal or pre-menopausal treated with goserelin.

Chemotherapeutic (e.g. cytotoxic) or hormonal agents or immunotherapeutic agents that can be co-administered with poziotinib include but are not limited to those agents that are more effective (e.g., more potent in inhibiting cancer cell growth) when co-administered with poziotinib.

In one embodiment, the determination of the patient's receptor status may be done by immunohistochemistry or in-situ rehybridization.

Additionally, administration of 1-(4-((4-((3,4-dichloro-2-fluorophenyl)amino)-7-methoxyquinazolin-6-yl)oxy)piperidin-1-yl)prop-2-en-1-one as a pharmaceutically acceptable acid may be appropriate.

Poziotinib can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, poziotinib may be systemically administered, e.g., orally, in combination with a pharmaceutically acceptable vehicle such as an inert diluent or an assimilable edible carrier. It may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Poziotinib may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound(s) in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, poziotinib may be administered to the skin as compositions or formulations, in combination with a dermatologically acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of poziotinib can be determined by comparing in vitro activity, and in vivo activity in animal models. Methods for the extrapolation of effective dosages in mice, and other animals, to humans are known to the art; for example, see U.S. Pat. No. 4,938,949. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

Poziotinib can be conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

In some embodiments, poziotinib can be co-administered with one or more other active therapeutic agents. Co-administration of poziotinib with one or more other active therapeutic agents generally refers to simultaneous or sequential administration of poziotinib and one or more other active therapeutic agents, such that therapeutically effective amounts of the compounds disclosed herein and one or more other active therapeutic agents are both present in the body of the patient. In some embodiments, poziotinib can be co-administered with one or more active therapeutic agents by combining the compounds disclosed herein with the other therapeutic agents in a unitary dosage form for simultaneous or sequential administration to a patient. Thus, this combination therapy may be administered as a simultaneous or sequential regimen. When administered sequentially, the combination may be administered in two or more administrations.

The invention will now be illustrated by the following non-limiting Examples.

### EXAMPLES

### Example 1

Poziotinib was purchased from a commercial source (Selleck Chem catalog number S7358). Assays were conducted using standard techniques that are known in the literature including those described in US Patent Publication U.S. 2015/0342909.

### Inhibition Assays

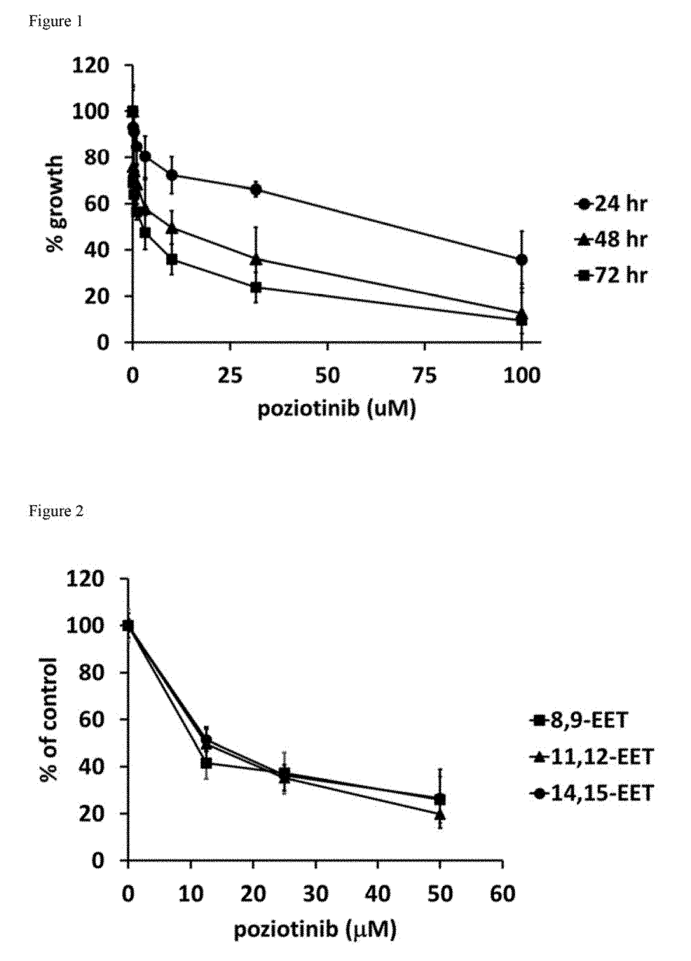

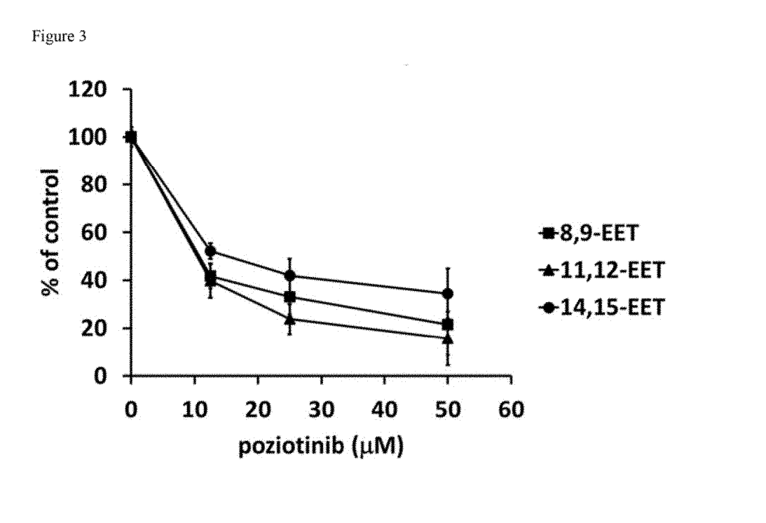

Poziotinib was tested for inhibition of proliferation of the MCF-7 (HER2 negative) cell line in complete media. The IC50 for inhibition of growth was determined to be approximately 3 μM (FIG. 1). Poziotinib was tested for inhibition of CYP2C8 supersome-mediated EET biosynthesis. The IC50 for inhibition of EET synthesis was determined to be approximately 10 μM (FIG. 2). Poziotinib was tested for inhibition of CYP3A4 supersome-mediated EET biosynthesis. The IC50 for inhibition of EET synthesis was determined to be approximately 10 82 M (FIG. 2).

### Measurement of OCR and ECAR

Cells were maintained in growth medium consisting of 10% FBS at 37° C. with 5% CO2 and seeded at 30,000 cells per well in XF24-well cell culture microplates (Seahorse Bioscience, North Billirica, Ma.). Concentrated stocks of poziotinib were prepared in DMSO. Poziotinib was diluted to 10× working concentration in XF assay medium (a non-buffered medium including 2 mM L-glutamine but no sodium bicarbonate (buffering agent), glucose, or sodium pyruvate). Assays were performed in the XF Extracellular Flux Analyzer (Seahorse Bioscience), which measures uptake and excretion of metabolic end products in real time. Oxygen consumption rate (OCR) and extracellular acidification rate (ECAR) were measured using an XF Assay Kit. OCR is reported in pmoles/minute and ECAR in mpH/minute. Before analysis, the cells were switched from culture medium to XF assay medium. After baseline measurements, 75 μl of poziotinib prepared in assay medium was injected into each well to reach final working concentrations. After addition of poziotinib, OCR and ECAR were measured at 18-minute intervals. There were 5 replicates performed for each data point.

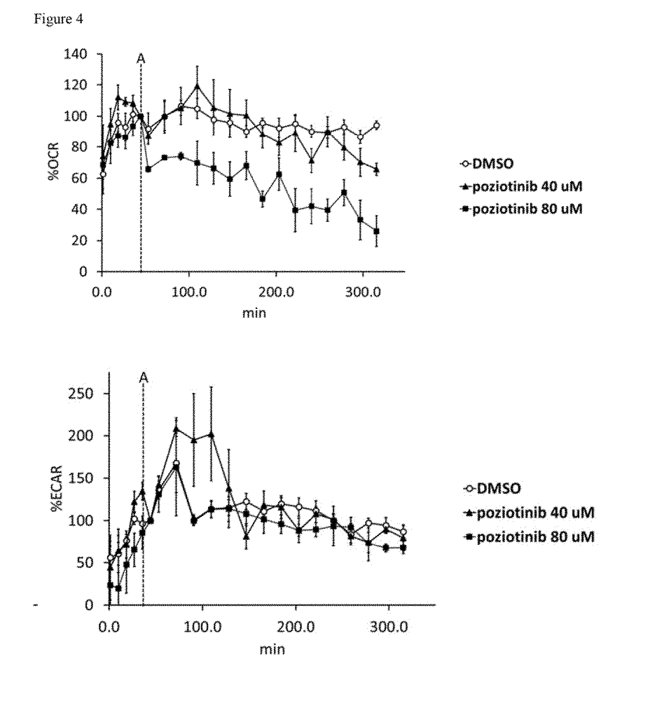

OCR was reduced by poziotinib vs. DMSO and was significant at the endpoint of A+270 minutes. OCR endpoint poziotinib 40 uM vs. DMSO P=0.0286, by t test; poziotinib 80 uM vs. DMSO P=0.0001. Results are normalized to the 6^{th} measurement DMSO control and expressed as mean ±S.D. (n=5) (FIG. 4).

### Example 2

The following illustrate representative pharmaceutical dosage forms, containing Poziotinib; 1-(4-((4-((3,4-dichloro-2-fluorophenyl)amino)-7-methoxyquinazolin-6-yl)oxy)piperidin-1-yl)prop-2-en-1-one or a pharmaceutically acceptable salt thereof (Compound X), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for treating breast cancer in an individual in need thereof comprising:
(a) determining hormone receptor status of at least two of the estrogen receptor, progesterone receptor (PR) and human epidermal growth factor receptor-2; and (b) administering to the individual an effective amount of a composition comprising poziotinib or a composition thereof.

2. The method of claim 1, wherein the status of at least two of the receptors is negative.

3. The method of claim 1, wherein the status the status of all receptors is negative.

4. The method of claim 1, wherein the status of the estrogen receptor is positive and the status of at least one of progesterone or human epidermal growth factor receptor-2 is negative.

5. The method of claim 1, wherein poziotinib is administered at doses ranging from 5-100 mg.

6. The method of claim 4, wherein poziotinib is administered at doses ranging from 10-750 mg.

7. The method of claim 1, further comprising the step of administering to said patient one or more additional agents selected from the group consisting of a cytotoxic agent, a hormonal agent, a immunotherapeutic agent, and an agent that inhibit the biosynthesis of one or more epoxyeicosatrienoic acids (EET).

8. The method of claim 7, further comprising administering to the patient one or more additional immunotherapeutic agents.

9. The method of claim 7, wherein the cytotoxic agent is selected from the group consisting of an antimetabolite, a mitotic inhibitor, alkylating agent, a platinum-based antineoplastic drug, an mTOR inhibitor, a VEGF inhibitor, an aromatase inhibitor and a CDK4/6 inhibitor.

10. The method of claim 8, wherein the antimetabolite is selected from the group consisting of Capecitabine, 5-Fluorouracil, Gemcitabine, Pemetrexed, Methotrexate, 6-Mercaptopurine, Cladribine, Cytarabine, Doxifludine, Floxuridine, Fludarabine, Hydroxycarbamide, decarbazine, hydroxyurea, and asparaginase.

11. The method of claim 9, wherein mitotic inhibitor is selected from eribulin mesylate, vindesine, vintafolide, 2-methoxyestradiol, trastuzumab emtansine, crolibulin, lorvotuzumab mertansine, topotecan/vincristine combinations, fosbretabulin tromethamine, estramustine phosphate sodium, vincristine, vinflunine, vinorelbine, cabazitaxel, vinblastine, epothilone A, patupilone, ixabepilone, Epothilone D, paclitaxel, docetaxel, discodermolide, eleutherobin, and pharmaceutically acceptable salts thereof or combinations thereof.

12. The method of claim 1, wherein the hormone receptor status of said receptors and is determined by immunohistochemistry.

13. A method for treating ER positive HER2 negative (ER+HER2−) breast cancer, triple negative breast cancer, or ER positive HER2 positive (ER+HER2+) breast cancer in a human patient with ER+HER2− breast cancer, triple negative breast cancer, or ER+HER2+ breast cancer comprising administering to the human patient an effective amount of Poziotinib.

14. The method of claim 13, wherein the method further comprises determining the HER2 status of the breast cancer in the human patient prior to the administration of Poziotinib.

15. The method of claim 13, for treating ER positive HER2 negative (ER+HER2−) breast cancer or triple negative breast cancer in a human patient with ER+HER2− breast cancer or triple negative breast cancer.

16. The method of claim 13, for treating ER positive HER2 negative (ER+HER2−) breast cancer in a human patient with ER+HER2− breast cancer.

17. The method of claim 13, further comprising administering to the patient one or more additional agents selected from hormonal agents, immunotherapeutic agents, chemotherapeutic agents or agents that inhibit the biosynthesis of one or more epoxyeicosatrienoic acids (EETs).

18. The method of claim 13, further comprising administering to the patient one or more additional immunotherapeutic agents.
